(19) United States
(12) Patent Application Publication
(10) Pub. No.: US 2019/0048193 A1
(43) Pub. Date: Feb. 14, 2019

(54) Hydrogel, Production, And Medical Device Comprising An Electrode Coated Therewith

(71) Applicant: Leonh. Lang, Innsbruck (AT)

(72) Inventors: Benjamin Naier, Innsbruck (AT); Herwig Schottenberger, Patsch (AT)

(21) Appl. No.: 15/761,432

(22) PCT Filed: Sep. 15, 2016
(86) PCT No.: PCT/EP2016/001553

§ 371 Date: Mar. 20, 2018

(30) Foreign Application Priority Data
Sep. 21, 2015 (DE) 10 2015 012 381.5

(51) Int. Cl. C08L 101/14; C08K 5/092; C08L 101/02

(52) U.S. Cl. CPC C08L 101/14; A61B 5/0408; C08L 101/025; C08K 5/092

(57) ABSTRACT

The invention relates to a hydrogel comprising cross-linked copolymer chains, wherein some of the repetitive units of the copolymer are based on functionalized monomers that contain both a polymerizable group and a complexing group, wherein the functionalized monomers further contain a cationic group, and wherein the hydrogel comprises anions corresponding to said cationic groups.

The invention relates to a hydrogel comprising cross-linked copolymer chains, wherein some of the repetitive units have a special functional profile. The invention further relates to a method of manufacturing such a hydrogel and to a medical device comprising an electrode coated with such a hydrogel.

A hydrogel is a three-dimensional network of polymer chains swollen with water. The swelling with water results in a considerable volume increase, but with the material cohesion of the polymer chains not being lost.

Hydrogels are used in various medical and non-medical fields, for example as a contact film on medical electrodes.

Hydrogels that are suitable for use as a contact film on medical electrodes should have high electrical conductivity. They should furthermore adhere well to the surface of the metallic electrode, whereas the adhesion at the fabric should admittedly be present to a certain degree, but should simultaneously not be too strong so as to be able to release the electrode without residue and without the risk of injury after the application. A high water absorption capacity can likewise be desirable in some cases in order e.g. to increase the cooling properties. In summary, it is therefore desirable to be able to monitor the electrical profile, the adhesion profile, and the swelling behavior in addition to other properties.

A hydrogel for use as a contact film on medical electrodes is known from DE 197 32 664 A1. This hydrogel comprises cross-linked copolymer chains, with some of the repetitive units of the copolymer being based on functionalized monomers that have both a polymerizable group and a complexing group.

It is the aim of the present invention to provide a hydrogel that has an improved electrical profile, adhesion profile, and swelling behavior and that offers a better possibility of setting these parameters. It should, however, simultaneously be achieved that the synthesis of the hydrogel remains comparatively simple.

Against this background, the invention relates to a hydrogel comprising cross-linked copolymer chains, with some of the repetitive units of the copolymer being based on functionalized monomers that have both a polymerizable group and a complexing group. Provision is made in accordance with the invention that the functionalized monomers furthermore have a cationic group and that the hydrogel comprises anions corresponding to these cationic groups.

The cationic group increases the hydrophilia of the copolymer and thus has an influence on the swelling behavior, the water absorption capacity, and the electrical conductivity. The adhesion on different surfaces and the electrical profile can be set using the anion. The ion pair furthermore permits the introduction of pH buffers, the introduction of passivation means, the introduction of active ingredients, and the introduction of functional anions. Despite these improved properties, the complexity of the manufacture of the hydrogel does not increase or only increases slightly.

In an embodiment, the cationic group comprises a quaternary nitrogen atom. Suitable such cationic groups comprises ammonium groups, for example those of the amine type, and N-substituted groups, for example N-alkylated heteroaromatic groups. Suitable such heteroaromatic groups comprise azolium groups, for example N-alkylated or N,N-dialkylated imidazols.

In an embodiment, the functionalized monomers comprise exactly one cationic group.

In an embodiment, the polymerizable group comprises a polymerizable ethylenic functionality, with it preferably being a substituted or unsubstituted vinyl group, allyl group, (meth)acrylate group or (meth)acrylamide group.

In an embodiment, the functionalized monomers comprise exactly one polymerizable group.

In an embodiment, the complexing group is a thiol, a phosphonic acid ester, an alkyne, a 1,3-dicarbonyl, an enamine, a dithiol, a triazole, a tetrazole, a carboxylic acid hydride, a cyanide, a chelate-forming amine, a diamine or a polyamine (the bonding group can also be the carbon atom of an NHC ligand).

In an embodiment, the functionalized monomers comprise exactly one complexing group.

In an embodiment, the anion is chloride, iodide, bromide, aryl sulfonate, alkyl sulfate, sulfate, aryl phosphate, alkyl phosphonate, monoalkyl phosphate, dialkyl phosphate, hydrogen phosphate, phosphate, hexafluorophosphate, hydrogen carbonate, carbonate, carbamate, alkyl carbonates, triflate or carboxylate.

If the anion has a side chain, such as can be the case with said hydrocarbon-modified anions, in particular alkylated and/or arylated anions, the desired hydrophilia or hydrophobia can additionally be modified. In accordance with the invention, the adhesive behavior can therefore not only be influenced by the selection of the monomers of the polymer network, but also by the selection of the anion.

It is furthermore possible to select the anions such that they have a positive effect on the durability of the electrode. For example, phosphonates or phosphates can promote the formation of a passivation coating or of an adhesion-promoting coating at the surface of an aluminum electrode or of another metal electrode.

Benzoates and salicylates can be used as anions, for example, in order likewise to extend the (microbiological) durability of electrodes.

In an embodiment, the anion is a complex-forming anion, for example citrate, malate, ethylenediaminetetraacetate, 2-phospho-L-ascorbate, or imidodiacetate. Such anions can inter alia have an influence on the adhesive profile, the electrical performance, and the storage life of the hydrogel.

In an embodiment, the anions are not bound to the copolymer in a covalent manner.

In an alternative embodiment, however, provision can be made that the at least one portion of the anions is bound to the copolymer chains in a covalent manner. For example, some of the repetitive units of the copolymer are based on anionic monomers that comprise a polymerizable group and an anionic group. Suitable polymerizable groups correspond to those groups that were discussed in connection with the functionalized monomers. Suitable anionic groups comprise the groups discussed above in connection with the free anions. Anions that are formed by splitting off an acidic proton are particularly preferred. Examples of suitable anionic monomers therefore comprise anionic derivatives of the (meth)acrylate or (meth)acrylamide, for example derivatives of the (meth)acrylate or (meth)acrylamide that include at least one sulfate group, sulfonate group, phosphonate group, phosphate group, carbonate group, carbamate group, triflate group, or carboxylate group, in particular a sulfonate group. Examples comprise acrylamido-2-methylpropanesulfonate or 3-(acryloyloxy)-1-propanesulfonate.

In an embodiment, the hydrogel comprises a combination of two or more different anions.

In an embodiment, the functionalized monomers have between 8 and 50 heavy atoms, and preferably between 10 and 30 heavy atoms. A heavy atom is understood as all atoms except for hydrogen in the present case.

In an embodiment, the molar mass of the functionalized monomers is between 100 and 3500 g/mol, preferably between 130 and 1000 g/mol.

In an embodiment, the functionalized monomers are ionic liquids. This means that the functionalized monomers are liquid, without being dissolved in a solvent, at a temperature that is below 100° C., and preferably at a temperature of 25° C. Ionic liquids are frequently themselves used as solvents, which can represent a further advantage of systems in accordance with the invention.

In an embodiment of the functionalized monomer, the cationic group is between the polymerizable group and the complexing group. The constitution of the functionalized monomer is therefore such in this embodiment that the preferably single chain of covalent bonds extending between the polymerizable group and the complexing group runs through the cationically charged center, for example through the positively charged nitrogen atom or the ring containing nitrogen and having the delocalized positive charge. Such monomers are obtained, for example, in that reactants comprising a polymerizable group and an amino group or an imino group or a heterocycle containing nitrogen are quaternized, with the complexing group being added as part of the quaternization of the nitrogen.

Suitable functionalized monomers comprise quaternized derivatives of N,N-dimethylaminopropyl(meth)acrylamide, N,N-dimethyl-3-aminopropyl(meth)acrylate, 2-(dimethylamino)ethyl(meth)acrylamide, 2-(dimethylamino)ethyl(meth)acrylate, vinylimidazole, vinylpyridine or vinylpyrrolidine, with the complexing group being added as part of the quaternization of the nitrogen.

Provision can be made in an embodiment that the quaternization occurs by the addition of glycidyl acrylate or by a nucleophile addition to a suitable complexing agent.

In an embodiment, the polymerizable functionality is produced by conversion using an acid chloride such as (meth)acrylic acid chloride.

The manufacture of suitable functionalized monomers is described, for example, in the following publications. Schottenberger et al., Dalton Trans., 2003, 4275-4281 describes the synthesis of 3-(prop-yn-1-yl)-1-vinyl-1-H-imidazolium bromide and 3-(prop-yn-1-yl)-1-allyl-1-H-imidazolium bromide. Their ability to form metal complexes is additionally documented. In Shapalov et al., J. Polym. Sci. A Polym. Chem., 2009, 4245-4266, the synthesis of 1-vinyl-3-(diethoxyphosphoryl)-propylimidazolium bromide and from this the synthesis of 1-vinyl-3-(diethoxyphosphoryl)-propylimidazolium-bis(trifluromethyl sulfonyl) imide is described.

Specific examples of functionalized monomers, for example, comprise the following compounds:

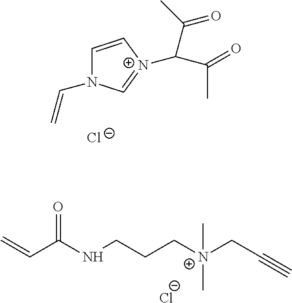

In an embodiment, the copolymer comprises two or more types of repetitive units that are based on different functionalized monomers.

In an embodiment, a further portion of the repetitive units of the copolymer is based on additional monomers that have a polymerizable group, but that differ from the functionalized monomers. These further monomers preferably do not have either a complexing group or a cationic group, but at least not both a cationic group and a complexing group.

In an embodiment, the polymerizable group of the additional monomers comprises a polymerizable ethylenic functionality. Preferred polymerizable groups comprise substituted or unsubstituted vinyl groups, allyl groups, (meth)acrylate groups, or (meth)acrylamide groups.

In an embodiment, the functionalized monomers comprise exactly one polymerizable group.

Examples of suitable additional monomers comprise (meth)acrylic acid and (meth)acrylic acid derivatives, inter alia acrylic acid, methacrylic acid, 3-sulfopropylacrylate, hydroxyethyl(meth)acrylate and (poly)ethylene glycol(meth)acrylate.

Further examples of suitable monomers comprise (meth)acrylamide and (meth)acrylamide derivatives, inter alia 2-acrylamido-2-methylpropane sulfonic acid (AMPS), 2-acrylamido-2-Methylpropane sulfonate, N,N-dimethyl aminoethyl acrylamide (DMAEAA), N,N-dimethyl aminopropyl acrylamide (DMAPAA), (3-acrylamidopropyl)trimethyl ammonium chloride, N-hydroxyethyl acrylamide (HEAA), 2-hydroxyethyl methacrylate (HEMA), dimethyl acrylamide (DMAA), propargyl acrylate, N-isopropyl acrylamide (NIPAM) and N-tert-butyl acrylamide (t-BAA).

Suitable additional monomers can be covered by one or more of the following definitions in an embodiment.

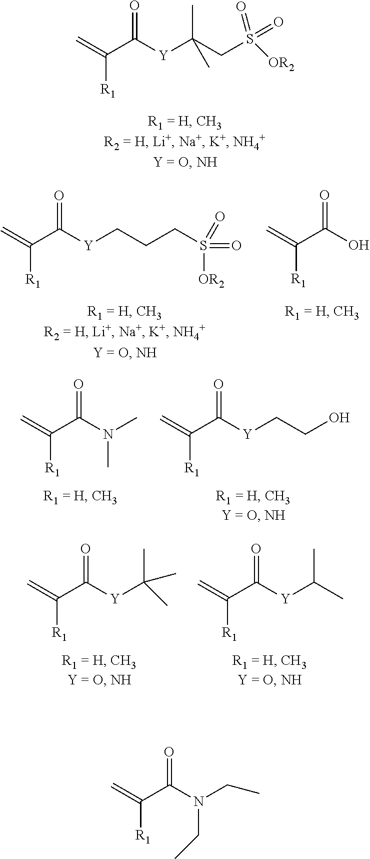

In an embodiment, the copolymer comprises two or more types of repetitive units that are based on different functionalized monomers.

The hydrogel can, for example, comprise repetitive units based both on (meth)acrylic acid and on (meth)acrylamide.

In an embodiment, the copolymer furthermore comprises cross-linked repetitive units that are based on cross-linkable monomers that have more than one polymerizable group, and preferably exactly two, exactly three, or exactly four polymerizable groups.

The cross-linked repetitive units serve the covalent cross-linking of individual polymer chains of the hydrogel.

In an embodiment, the polymerizable group of the additional monomers comprises a polymerizable ethylenic functionality. Preferred polymerizable groups comprise substituted or unsubstituted vinyl groups, allyl groups, (meth)acrylate groups, or (meth)acrylamide groups. All the polymerizable groups of the cross-linkable monomers are preferably identical.

In an embodiment, the cross-linkable monomers do not comprise any complexing or cationic groups.

Examples of suitable cross-linkable monomer units comprise methylene methylbis(meth)acrylate, ethylene bisethyl(meth)acrylate, (poly)ethylene glycol di(meth)acrylate, glycerol di(meth)acrylate, glycerol tri(meth)acrylate, 1,9-nonanediol di(meth)acrylate and trimethylolpropanethoxylate triacrylate.

In an embodiment, the polymer structure of the hydrogel can exclusively comprise repetitive units that are based on functionalized, additional, and cross-linkable monomers.

In an embodiment, the different repetitive units are statistically distributed in the copolymer.

In an embodiment, the hydrogel furthermore includes multivalent alcohols. They serve as plasticizers and as a hygroscopic agent.

Examples of suitable multivalent alcohols comprise ethylene glycol, propylene glycol, butanediol, glycerol, pentaerythritol, sorbitol, (poly)ethylene glycol (for example polyethylene glycol 300, polyethylene glycol 400 or polyethylene glycol 600), (poly)polypropylene glycol, polyglycerol, polyoxyethylene ether and polyglyceryl ether.

In an embodiment, the portion of the repetitive units based on functionalized monomers in the hydrogel amounts to 0.1 to 40 wt. %. Preferred ranges comprise 0.5 to 20 wt. % and 1 to 10 wt. %.

In an embodiment, the portion of the repetitive units based on additional monomers in the hydrogel amounts to 5 to 50 wt. %. Preferred ranges comprise 10 to 40 wt. % and 15 to 35 wt. %.

In an embodiment, the portion of the cross-linked repetitive units in the hydrogel amounts to 0.01 to 5 wt. %. Preferred ranges comprise 0.02 to 1 wt. % and 0.05 to 0.4 wt. %.

In an embodiment, the portion of the multivalent alcohols in the hydrogel amounts to 5 to 60 wt. %. Preferred ranges comprise 10 to 50 wt. % and 15 to 45 wt. %.

The indicated values relate to the total mass of the hydrogel, that is to the dry weight of the hydrogel and the mass of the water absorbed in the hydrogel.

In an embodiment, the hydrogel comprises the polymer structure, water, the anion, the multivalent alcohols, and possible contaminants whose proportion is, however, less than 3 wt. % of the total mass of the hydrogel. In an embodiment, additional fillers can be included.

Against the initially named background, the invention furthermore relates to a method of manufacturing a hydrogel in accordance with the invention, wherein the cross-linked copolymer chains are manufactured by a radical chain polymerization of the functionalized and preferably furthermore additional and/or cross-linkable monomers.

The functionalized monomers can be introduced into the reaction solution as monomers, oligomers or polymers, optionally copolymers or cooligomers.

In an embodiment, the polymerization is photoinitiated, preferably by means of a photoinitiator.

The light used is preferably UV light.

The use of photoinitiators that respond to light in the UV range is particularly preferred. Suitable photoinitiators, for example, comprise 2-hydroxy-2-methyl-1-phenyl-propane-2-one, 2-hydroxy-1-[4-(2-hydroxyethoxy)phenyl]-2-methylpropane-1-one, 2,2-dimethoxy-2-phenyl acetophenone, 1-[4-(2-hydroxyethoxy)-phenyl]-2-hydroxy-2-methyl-1-propane-1-one, 1-hydroxycyclohexyl phenyl ketone or trimethylbenzoyldiphenylphosphinoxide. The proportion of the photoinitiators in the polymerizable starting mass can amount to between 0.05 and 3 wt. %. Preferred ranges comprise 0.01 to 1.5 wt. %, 0.1 to 0.8 wt %, or 0.15 to 0.7 wt. %.

In an alternative embodiment, curing in the visible range is also conceivable Photoinitiators suitable for such a curing comprise, for example, bis-(4-methoxybenzoyl)diethylgermane.

In an embodiment, a radiation initiation or a photoinitiation can also take place directly, i.e. without a photoinitiator, for example by using high energy radiation such as y radiation or E-beam.

Alternatively, the polymerization can also be initiated chemically or thermally. For example, an initiation can take place using an organic peroxide such as benzoylperoxide, an inorganic peroxo system such as potassium peroxo disulfate, or an azo-based initiator such as azobisisobutyronitrile. A Fenton polymerization is also suitable for use as part of a method in accordance with the invention.

Anionic or cationic polymerization is also conceivable.

In an embodiment, the complexing group is protected by a protective group during the manufacture of the copolymer. Secondary reactions such as hydrolysis, a hydrolytic decomplexing, or an oxidation can thus be avoided, for example. The protective group can be removed on the mixing of the monomers or after the manufacture of the copolymer.

In an embodiment, the cationic group of the functionalized monomer can be protected. The protection can take place by a thioketone, for example.

In an embodiment, at least some metal ions are already bound to the complexing group of functionalizing monomers before the polymerization. Possible reactions of these metal ions comprise the catalysis of the polymerization reaction, a metal-initiated polymerization, or a reaction such as described in U.S. Pat. No. 4,846,185.

In an embodiment, the hydrogel can be at least partially saturated after the polymerization by a process with metal ions.

The multivalent alcohol is preferably added on the manufacture of the reaction mixture of the monomers.

In an embodiment, the functionalized monomers, which are an ionic liquid, themselves serve as a solvent for the polymerization reaction.

In an embodiment, the method comprises a process of an at least partial replacement of the anions after a completed manufacture of the cross-linked polymer chains.

This process can, for example, comprise the at least partial replacement of chloride ions with a different anion.

A suitable example for the implementation of such a process comprises the so-called acetone method (under Finkelstein conditions) in which an acetonic solution of any desired sodium salt penetrates into the network. Due to the poor solubility of sodium chloride in acetone, sodium chloride is precipitated and an anion metathesis takes place. The metathesis reaction here can take place by at least one second hydrogel film or by addition of a solution or by an anion supplied from the outside. The use of other known methods of anion replacement and of salt metathesis is likewise conceivable.

A possible area of use of the hydrogels in accordance with the invention is their use as a contact film on medical electrodes.

Against this background, the invention furthermore relates to a medical device comprising at least one electrode, wherein at least a part of this electrode is coated with a hydrogel in accordance with the invention.

In an embodiment, the medical device is a defibrillator, an ECG machine, an EEG machine, a TENS machine, an iontophoresis machine, an electric scalpel, or an electric stimulation machine.

In an embodiment, the electrode is a defibrillation electrode, an ECG electrode, an EEG electrode, a TENS electrode, a iontophoresis electrode, a neutral electrode, or a wound electrode.

The use of hydrogels in accordance with the invention is not, however, restricted to medical electrodes. Further possibilities of use comprise a use in glucose sensors, in charged membranes, in metal removal, and for example in heavy metal removal from aqueous systems, or as an adhesive agent at surfaces, metallic surfaces for example.

Further details and advantages result from the Figures and embodiments discussed in the following. There are shown in the Figures:

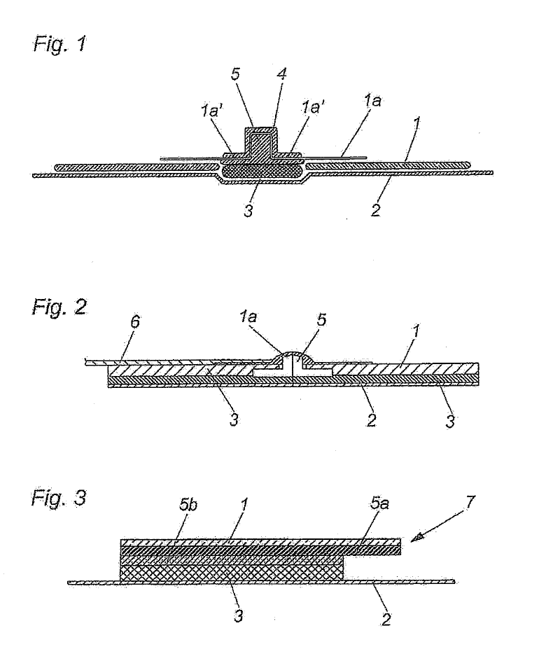

FIG. 1: a cross-section of an ECG electrode that is coated with a hydrogel in accordance with the invention;

FIG. 2: a cross-section through a different ECG electrode that is likewise coated with a hydrogel in accordance with the invention;

FIG. 3: a cross-section through yet another ECG electrode that is likewise coated with a hydrogel in accordance with the invention;

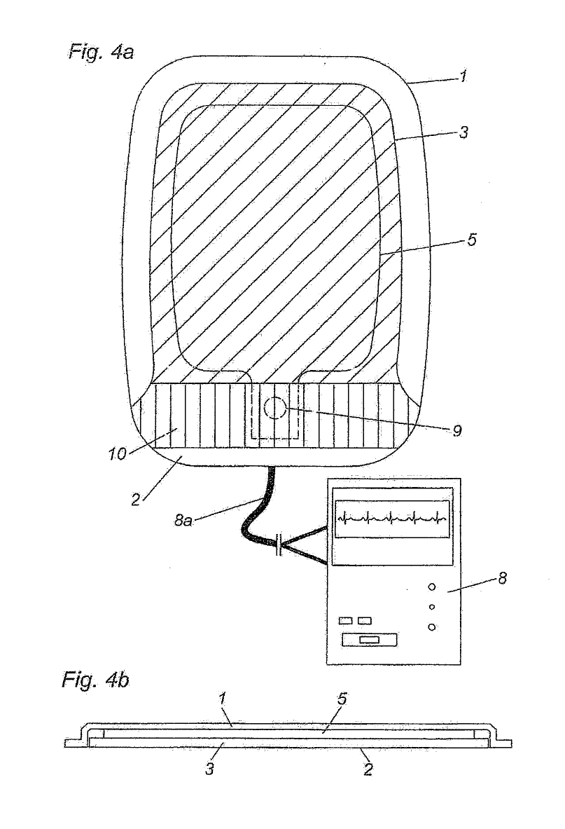

FIG. 4: views of a defibrillator in accordance with the invention;

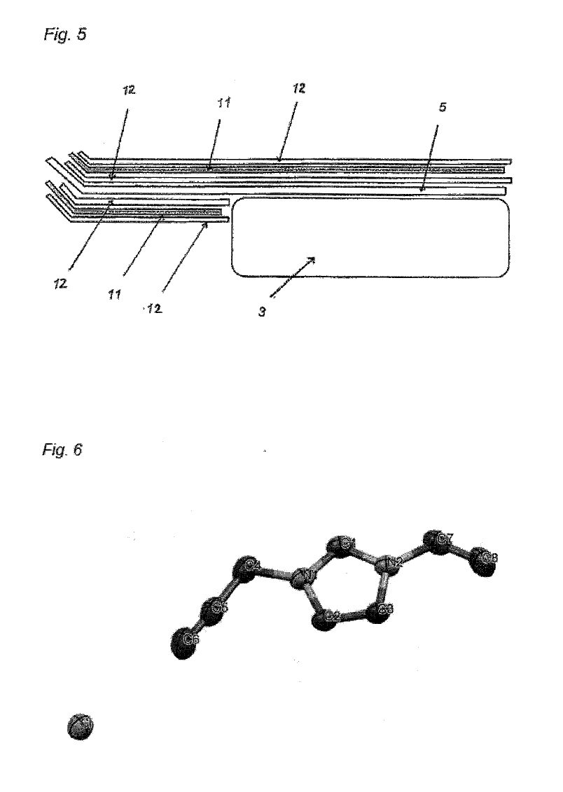

FIG. 5: a cross-section through a further biomedical electrode that is coated with a hydrogel in accordance with the invention; and

FIG. 6: a crystalline structure of the 3-(prop-2-yn-1-yl)-1vinyl-1H-imidazolium chloride of synthesis example 3.

The biomedical electrode (ECG electrode with button) shown in FIG. 1 has a carrier, for example from plastic or from a plaster material. This carrier is adhesively coated on its lower side facing the skin. A masking film 2 protects, on the one hand, the adhesive layer on the lower side of the carrier 1 and, on the other hand, the conductive hydrogel 3 in accordance with the invention described below in detail. This masking material 2 is removed and the electrode is bonded on before use.

A label 1a, preferably consisting of a stiffer plastic film, is applied, for example adhesively bonded, to the upper side of the carrier 1. This label 1a forms a part of the carrier and carries the electrical connector element 4 in the form of a button composed of stainless steel or carbon. This connector element is in electrically conductive contact with an electrical lead element 5, wherein the carrier part or the label 1a is arranged at the points 1a′ between the electrical lead element 5 and the electrical connector element 4 and thus holds the connector button mechanically at the ECG electrode.

The gel 3 in accordance with the invention can be designed, for example, as a solid gel that is configured in a rigid manner. It can, however, also be present as a thickener in a liquid gel that is held in a sponge.

The electrical lead element 5 that is electrically conductively connected to the connector element (button 4), is in contact with the conductive gel at itself lower side and is electrically connected to it there.

The individual parts disposed above one anther are shown spaced apart in FIG. 1. This only serves for a better illustration. In reality, the layers drawn separately above one another are connected to one another, with the protective film 2 being removed before use.

In the embodiment in accordance with FIG. 2, it is a precabled ECG electrode. The same reference numbers designate the same or similar parts as in FIG. 1. The main difference substantially comprises an electrically conductive cable 6 that is fixedly connected to the electrode being provided instead of the connector button 4, while a cable to an ECG evaluation device, not shown, is connected to the connector button 4. The cable 6 or the strand located therein is electrically connected to the lead element 5 that is in turn in contact with the conductive gel at the lower side. The label 1a at the upper side of the carrier 1 can be configured as electrically conductive and can in so doing establish an electrical contact between the cable 6 and the electrical lead element 5. It is, however, also possible that this carrier part 1a (label) comprises non-conductive material. The cable 6 or the strand located therein then has to be electrically connected to the electrical lead element 5 in another manner.

The embodiment shown in FIG. 3 is an ECG electrode with a lug contact. The same reference numerals again designate the same or similar parts as in FIGS. 1 and 2.

A laterally projecting lug 7 that has the carrier material 1, for example a relatively rigid strip of paper of PET, serves as a connector element here. This carrier strip 1 or the lug 7 has an electrically conductive film 5a at the lower side that is connected to a connector terminal. The film 5a can, for example, consist of carbon or can predominantly have this as the electrically conductive component. Suitable such possibilities and alternative possibilities comprise a carbon film and silver lacquer, a carbon film filled with silver, a tin film, or generally a depolarizing film. In the present case, a depolarizing film is to be understood as a film that is able to provide both oxidized species and reduced species simultaneously in combination with a hydrogel. This film 5a is connected to a further film 5b. The film 5b can, for example, comprise an electrically conductive lacquer, that is a different material than the film 5a. It is, however, also possible that the film 5b is completely missing. Ultimately, the two films 5a and 5b together form the electrical lead element that is connected to the conductive gel at the lower side. This lies on the skin after removal of the film 2 and is, in a similar manner to the gel in FIG. 2, also adhesive so that the electrode adheres to the skin.

The embodiment shown in FIGS. 4a and 4b relates to a defibrillation electrode. The same reference numerals again designate the same or similar parts.

The design is generally similar to that in the ECG electrodes in FIGS. 1 to 3. The area is lager to enable a current transfer from the defibrillation machine 8 to the human body.

The conductive gel 3 is preferably a solid gel. An electrical connection from the connector cable 8a via a rivet 9 to the lead element 5 takes place via a film 10.

In the embodiment shown in FIG. 5, an electrical lead element 5 in the form of a flexible film is provided. Here, a respective three films are printed on the upper side and lower side of the electrical lead element 5, and indeed a dielectric (insulating) film 12 as well as a respective electrically conductive shield film 11 disposed therebetween. It is above all possible with diagnosis electrodes thanks to this shielding to shield interfering electromagnetic radiations and other interference sources. The continuation of the lead element, starting from the region covered by the gel 3, simultaneously provides a shielded feed cable. The imprinting of the shielding is possible by a simple manufacture. A good shield effect can nevertheless be achieved.

Provision is now made in accordance with the invention that the conductive gel 3 represents one of the gels in accordance with the invention and is at least partially connected to the electrical lead element. The electrical lead element 5 can be passivated as protection against corrosion. The lead element is typically of layer form or film form. It can, however, also be present in fiber form.

It is also possible that the gel comprises different films, with the gel in accordance with the invention representing one or more films of this design. Another variant is that the gel is admittedly set up of one film, but is in turn made up of different gels, with the gel in accordance with the invention again being able to represent one or more parts of this film. A combination of these variants is also possible.

### LIST OF REFERENCE NUMERALS

1 carrier
2 cover film
3 hydrogel in accordance with the invention
4 connector element
5 electrical lead element
6 connector element
7 electrical connector element
8 defibrillator
9 rivet
10 film over which an electrical conduction takes place
11 shield element
12 dielectric film

The definition of individual reagents used in the following examples is collected in the following Table 1:

TABLE 1

| | |
|---|---|
| NaAMPS 50% | Sodium salt of the 2-acrylamido-2- |
| | methylproansulfonic acid (50% in H₂O) |
| PVP K 90 | Polyvinylpyrrolidone K90 |
| Irgacure 754 | Photoinitiator from Ciba |
| Esacure KTO 46 1:1 in EtOH | Photoinitiator from Lamberti |
| Tylose YP 300000 | Hydroxyethylcellulose |
| Lutensol XL 70 | Alkylpolyethylene glycolether from BASF |
| CN 3705 | Diacrylate triethanolamine from Sartomer |
| Irgacure 2959 | 1-[4-(2-hydroxy)-phenyl]-2-hydroxy- |
| | 2-methyl-1-propane-1-one |
| Esacure DP 250 | Photoinitiator from Lamberti |
| Irgacure 184 | 1-hydroxy-cyclohexyl-phenyl-ketone |
| Bardac 2270 | Didecyldimethylammonium chloride |

### SYNTHESIS EXAMPLE 1

### 3-(2,4-dioxopentane-3-yl)-1-vinyl-imidazoliumchloride

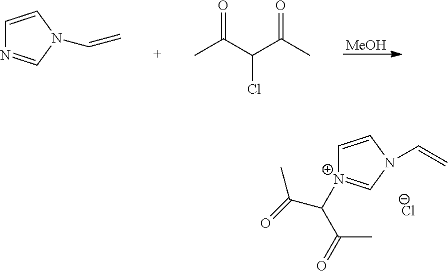

9.42 g vinylimidazole and a spatula tip of hydroquinone monoethyl ether are dissolved in 20 ml acetone to prepare a first solution. The solution is degassed with argon for 10 minutes. A second solution is prepared by dissolving 13.46 g 3-chloroacetylacetonate in 15 ml acetone. The second solution is slowly dripped into the first solution with external ice cooling. The combined solution is held under the external ice cooling for 2 hours and is subsequently stirred at room temperature for 4 hours. An extraction with water and chloroform subsequently takes place, with the water phase being washed three times with chloroform and the water being extracted at the rotary evaporator. 19.87 g product in the form of a yellow highly viscous liquid is obtained.

### SYNTHESIS EXAMPLE 2

### 3-(4-butyryl-5-oxooctyl)-1-vinylimidazolium Chloride

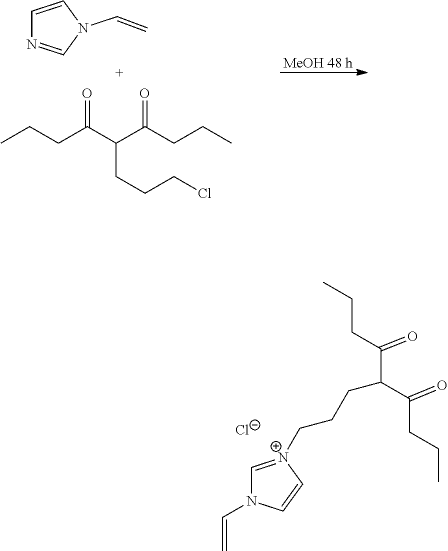

0.47 g of the liquid reactant vinylimidazole are presented in a vessel and 1.18 g diethyl (3-chloropropyl) malonate are added in 7.5 ml methanol. The mixture is stirred at room temperature for 48 hours. The solvent is subsequently extracted and the sample is dried under high vacuum. 93% of the theoretical yield of the product is obtained with the NMR spectra shown in the following: 1H NMR (300 MHz, neat) δ 7.94, 7.51, 7.17, 5.51, 4.96, 4.30, 3.71, 3.58, 2.16, 1.96, 1.33; 13C NMR (75 MHz, neat) δ 169.32, 136.86, 130.43, 130.23, 116.18, 100.79, 61.54, 51.42, 44.87, 30.44, 26.46, 14.16.

### SYNTHESIS EXAMPLE 3

### 3-(prop-2-yn-1-yl)-1vinyl-1H-imidazolium Chloride

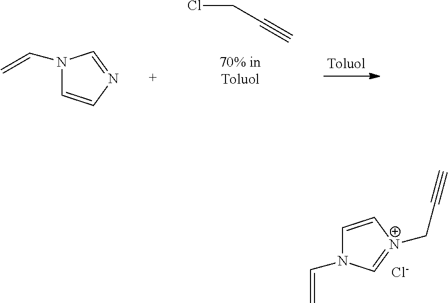

4.70 g vinylimidazole are dissolved in 15 ml toluol. 5.32 g propargyl chloride (70% in toluol) are subsequently added. The reaction solution is heated with backflow for 8 hours, with a white deposit being formed. The deposit is subsequently filtered off and washed with ether. 1.837 g (22% of the theoretical yield) of the product is produced in the form of a white powder having the NMR spectra shown in the following: 1H NMR (300 MHz, DMSO) δ 9.93, 9.92, 9.92, 8.41, 8.40, 8.39, 8.03, 8.03, 8.02, 7.48, 7.45, 7.43, 7.40, 6.10, 6.10, 6.05, 6.04, 5.45, 5.44, 5.42, 5.41, 5.33, 5.32, 3.94, 3.93, 3.92

An X-ray crystalline structure of the product is shown in FIG. 6.

### SYNTHESIS EXAMPLE 4

### 3-(2-(diethylamino)ethyl)-1-vinyl-1H-imidazolium Bromide

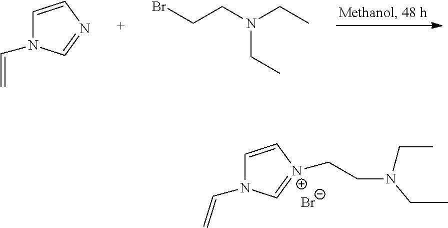

4.7 g vinylimidazole are presented in 40 ml methanol and are washed with argon. 9.00 g 2-bromo-N,N-diethylethylamine is slowly dripped in under external ice cooling and the reaction mixture obtained is stirred at room temperature for 48 hours. The solvent is subsequently removed at the rotary evaporator and the created white crystalline product was washed multiple times with diethyl ether. 11.34 g of the product are obtained.

### EMBODIMENTS 1 TO 3 AND COMPARISON EXAMPLE 4

At least one non-functionalized monomer is presented in water and at least one multivalent alcohol is slowly added. In another vessel, the functionalized monomer, which is an ionic liquid, a cross-linking monomer and a photoinitiator are mixed. Once a homogeneous solution of the second solution has been produced, it is slowly dripped into the parent solution while stirring. After combination of the solutions stirring takes place at 500 revolutions per minute for 20 minutes and the solution is degassed in an ultrasound bath for 15 minutes.

The polymerizable solution obtained in this manner is applied to a tin antimony film (98:2) with the aid of a film drawing machine. The layer thickness of the film amounts to 4 mm. The polymerizable solution is then hardened using a UV mercury vapor discharge lamp. An almost transparent adhesive hydrogel is produced.

The ingredients of the polymerizable solutions are collected in Table 2. Electrical measurement values and measurement values of the resulting hydrogels obtained as part of the delamination measurement are collected in Table 3.

TABLE 2

| | | | | | Type or function |
|---|---|---|---|---|---|
| | AB 1 | AB 2 | AB 3 | Cf. B 4 | of the ingredient |
| NaAMPS 50% in water | 63.74 | 20.27 | | 46.31 | Monomer |
| Acrylic acid | 1.35 | 15.30 | 11.32 | 0.51 | Monomer |
| Glycerin | 25.86 | 14.75 | 31.06 | 20.38 | Multivalent alcohol |
| Sorbitol 70% in water | | 14.00 | | | Multivalent alcohol |
| Triethanolamine | | 1.55 | 3.38 | | Base |
| Ammonium chloride | 1.59 | 1.46 | 2.20 | | Conducting salt |
| Water, demineralized | | 19.04 | 33.99 | 18.49 | |
| PVP K90 | 0.27 | | | | Thickener |
| 2-hydroxy-2-methyl-1-phenyl-proan-1-one | 0.21 | 0.20 | 0.33 | 0.37 | Photoinitiator |
| Irgacure 754 | | 0.12 | 0.17 | 0.19 | Photoinitiator |
| Ethyleneglycoldimethacrylate | 0.16 | | 0.24 | 0.13 | Cross-linker |
| Tylose ® 30000 YP | | 0.10 | 0.17 | 0.09 | Thickener |
| Caustic potash 47% in water | | 3.68 | 0.34 | 0.19 | Base |
| 3-acrylamido-N-(2-(diethoxyphosphoryl)ethyl)- | | 3.08 | | | Functionalized |
| N,N-dimethylpropane-1-aminium chloride | | | | | monomer |
| Trimethylolpropane triacrylate | | | | 0.09 | Cross-linker |
| Potassium tartrate tetrahydrate | 0.72 | | | | Conducting salt |
| Sodium oxalate | 0.30 | | | | Conducting salt |
| Tin oxalate | 0.01 | 0.01 | | | Conducting salt |
| 3-(2-(diethylamino)ethyl)-1-vinyl-1H- | 2.25 | | 5.59 | | Functionalized |
| imidazolium bromide | | | | | monomer |
| CN 386 (Monoacrylate triethanolamine | 1.01 | 2.23 | | 0.19 | Monomer |
| Sartomer) | | | | | |
| Irgacure 184 | 0.36 | 0.19 | | | Photoinitiator |
| 1-vinylpyrrolidone | 2.18 | | | 1.12 | Monomer |
| 4-hydroxybutylvinyl ether | | | | 1.22 | Monomer |
| Bardac 2270 | | | | 0.03 | Filler (biocide) |
| Potassium sodium tartrate | | | | 0.96 | Conducting salt |
| Sodium chloride | | | | 2.13 | Conducting salt |
| Hydroxyethylacrylamide | | 3.18 | 11.20 | 7.60 | Monomer |
| Magnesium acetate tetrahydrate | | 0.83 | | | Conducting salt |

TABLE 3

| Type of measurement value | AB 1 | AB 2 | AB 3 | Cf. B 4 |
|---|---|---|---|---|
| Delamination on tin antimony film | 2.52 | 1.55 | 2.15 | 1.03 |
| 98:2 [N] | | | | |
| Large signal impedance [Ohm] | 0.33 | 0.35 | 0.24 | 0.2 |
| DCO after 4 seconds [mV] | 94.6 | 6.90 | 22.9 | 93.7 |
| DCO after 60 seconds [mV] | 44.6 | 8.00 | 10.5 | 30.5 |

The impedance values and offset voltages were determined in accordance with IEC 60601-2-4.

The force required for the delamination of the hydrogel should be sufficiently high to prevent an unwanted peeling of the hydrogel. Values of 1 N or more are to be considered as sufficient with respect to the respective film. Small impedance values are preferred, with all the gels easily satisfying the requirement of the standard IEC 60601-2-4 of less than 3 ohms and all the values in the range of less than 1 ohm can be considered as very good. Small offset voltages are important, for example, for recording an ECG.

1. A hydrogel comprising cross-linked copolymer chains, with some of the repetitive units of the copolymer being based on functionalized monomers that have both a polymerizable group and a complexing group,
characterized in that
the functionalized monomers furthermore have a cationic group; and in that that the hydrogel comprises anions corresponding to these cationic groups.

2. A hydrogel in accordance with claim 1, characterized in that the cationic group is a quaternary nitrogen atom.

3. A hydrogel in accordance with claim 1, characterized in that the polymerizable group comprises a polymerizable ethylenic functionality.

4. A hydrogel in accordance with claim 1, characterized in that the complexing group is a thiol, a phosphonic acid ester, an alkyne, a 1,3-dicarbonyl, an enamine, a dithiol, a triazole, a tetrazole, a carboxylic acid hydride, a cyanide, a chelate-forming amine, a diamine or a polyamine.

5. A hydrogel in accordance with claim 1, characterized in that at least some of the anions are chloride, iodide, bromide, aryl sulfonate, alkyl sulfate, sulfate, aryl phosphonate, alkyl phosphonate, monoalkyl phosphonate, dialkyl phosphate, hydrogen phosphate, phosphate, hexafluorophosphate, hydrogen carbonate, carbonate, carbamate, alkyl carbonates, triflate or carboxylate.

6. A hydrogel in accordance with claim 1, characterized in that at least some of the anions are complex-forming anions.

7. A hydrogel in accordance with claim 1, characterized in that at least some of the anions are bound to the copolymer chains in a covalent manner.

8. A hydrogel in accordance with claim 1, characterized in that a further portion of the repetitive units of the copolymer is based on additional monomers that have a polymerizable group, but that do not further have both a complexing and a cationic group.

9. A hydrogel in accordance with claim 1, characterized in that the copolymer furthermore comprises cross-linked repetitive units that are based on cross-linkable monomers that have more than one polymerizable group.

10. A hydrogel in accordance with claim 1, characterized in that the hydrogel furthermore includes multivalent alcohols.

11. A hydrogel in accordance with claim 1 characterized in that the portion of the repetitive units based on functionalized monomers in the hydrogel amounts to 0.1 to 40 wt. %; and/or in that the portion of the repetitive units based on additional monomers in the hydrogel amounts to 5 to 50 wt. %; and/or in that the portion of the cross-linked repetitive units in the hydrogel amounts to 0.01 to 5 wt. %; and/or in that the portion of the multivalent alcohols in the hydrogel amounts to 5 to 60 wt. %.

12. A method of manufacturing a hydrogel in accordance with claim 1,
characterized in that
the cross-linked copolymer chains are manufactured by a radical chain polymerization of the functionalized monomers.

13. A method in accordance with claim 12, characterized in that the polymerization is photoinitiated.

14. A method in accordance with claim 12, characterized in that the method comprises a process of an at least partial replacement of the anions after a completed manufacture of the cross-linked polymer chains.

15. A medical device comprising at least one electrode,
characterized in that
at least a part of the electrode is coated with a hydrogel in accordance with claim 1.

16. A hydrogel in accordance with claim 1, characterized in that the cationic group is an ammonium group

17. A hydrogel in accordance with claim 1, characterized in that the cationic group is an amine type or an azolium group.

18. A hydrogel in accordance with claim 1, characterized in that the polymerizable group comprises a polymerizable ethylenic functionality that is a substituted or unsubstituted vinyl group, allyl group, (meth)acrylate group or (meth)acrylamide group.

19. A hydrogel in accordance with claim 1, characterized in that at least some of the anions are citrate, malate, ethylenediaminetetraacetate, 2-phospho-L-ascorbate, or imidodiacetate.

20. A hydrogel in accordance with claim 1, characterized in that the copolymer furthermore comprises cross-linked repetitive units that are based on cross-linkable monomers that have exactly two, exactly three, or exactly four polymerizable groups.
